(19) United States
(12) Patent Application Publication
(10) Pub. No.: US 2016/0228675 A1
(43) Pub. Date: Aug. 11, 2016

(54) Apparatus and Method to Form Tip Profiles

(71) Applicant: Syneo, LLC, West Palm Beach, FL (US)

(72) Inventors: Daniel A. Woodward, West Palm Beach, FL (US); Edward Ethen, West Palm Beach, FL (US); Manuel Ramos, Port St. Lucie, FL (US)

(21) Appl. No.: 15/018,940

(22) Filed: Feb. 9, 2016

Related U.S. Application Data
(60) Provisional application No. 62/113,870, filed on Feb. 9, 2015.

(51) Int. Cl. A61M 25/00

(52) U.S. Cl. CPC A61M 25/001

(57) ABSTRACT

An apparatus and method to thermally form a smooth profile tip on a polymer tube such as a catheter. The process employs a metal mold that contains the desired form. The polymer tube is inserted partially into a cool mold which is heated sufficiently to soften the plastic. The tube is then forced further into the mold until the form is completed. The tube is gripped with a clamp and inserted into the mold using a motorized linear stage. In addition to time and temperature control, the apparatus and method adds load cell based force feedback and a motorized linear stage with speed control and encoder based position feedback. The control software provides a multi-step process flow where each step includes user programmable force, distance, speed, temperature, and time. Users can define each parameter in each step to produce a desired result.

### PRIORITY CLAIM

In accordance with 37 C.F.R. 1.76, a claim of priority is included in an Application Data Sheet filed concurrently herewith. Accordingly, this present invention claims priority to U.S. Provisional Patent Application No. 62/113,870 entitled “APPARATUS AND METHOD TO FORM TIP PROFILES”, filed Feb. 9, 2015. The contents of the above referenced application is incorporated herein by reference.

### FIELD OF THE INVENTION

The field of the invention is related to the apparatus used to manufacture medical devices and, in particular, to an apparatus and method to thermally form a smooth profile tip on a polymer tube such as a catheter.

### BACKGROUND OF THE INVENTION

Prior art device control was typically limited to manually adjusted air pressure and digitally controlled temperature and time. By adding software controlled insertion force, distance, and speed a user can now control the process and determine if the resulting product is the same from cycle to cycle.

### SUMMARY OF THE INVENTION

An apparatus and method to thermally form a smooth profile tip on a polymer tube such as a catheter. The process typically uses a metal mold that contains the desired form. The tube is inserted partially into the cool mold which is then heated sufficiently to soften the plastic. The tube is then forced further into the mold until the form is completed. The mold and part are cooled to solidify the form and the completed part is removed from the mold. Typically the tube is gripped with a clamp and inserted into the mold using an air cylinder and manually controlled air pressure. Automatic process control is limited to temperature and time.

The apparatus provides process flexibility by allowing a user to manipulate more parameters and achieve output repeatability by controlling more parameters. By adding software controlled insertion force, distance, and speed the user has many more options for both controlling the process and determining if the resulting product is the same from cycle to cycle. The cooling cycle is terminated based on temperature assuring each part starts at the same temperature. The force to withdraw a part from the mold is also monitored and can provide feedback for corrective action before sticking in the mold becomes a problem. Each parameter can be individually manipulated in a user defined multi-step process sequence to produce the desired results. Namely, (1) the part can be inserted into the cooled mold until a user defined force is reached to ensure a consistent starting position, (2) mold/part preheat can be initiated for a user programmable time period, and (3) additional insertion(s) at distance and/or force can be programmed. The final insertion distance and force can be registered by the control computer and deviations used to detect process anomalies. Finally, the optional vision inspection module can measure the part for proper profile dimensions and detect defects such as flash and incomplete form.

An objective of the invention is to control an RF tipping and bonding process using an apparatus that is flexible and output repeatable.

Another objective of the invention is to provide a temperature control using a temperature feedback device.

Still another objective of the invention is to provide a dwell time control using digital timer in controller.

Another objective of the invention is to provide part insertion speed control using motor driven linear stage.

Still another objective of the invention is to provide part insertion force control using strain gage feedback device.

Yet still another objective of the invention is to provide part insertion depth control using encoder feedback.

Another objective of the invention is to provide a cooling temperature control temperature feedback device.

Still another objective of the invention is to provide part retraction speed control using motor driven linear stage.

Yet still another objective of the invention is to provide part retract force detection using strain gage feedback device.

Another objective of the invention is to provide an optional machine vision inspection of dimensions and quality.

Other objectives and further advantages and benefits associated with this invention will be apparent to those skilled in the art from the description, examples and claims which follow.

### BRIEF DESCRIPTION OF THE DRAWINGS

FIG. 1 is a right front perspective view of the tip forming apparatus;

FIG. 2 is a left front perspective view of the tip forming apparatus with a cover plate removed;

FIG. 3 illustrates the clamp axis encoder, lead screw and linear slide;

FIG. 4 illustrates the clamp is a perspective view of the clamp axis drive motor and limit sensors;

FIG. 5A illustrates a perspective view of the primary part clamp carriage assembly;

FIG. 5B is a cross sectional view of the clamp carriage of FIG. 5A;

FIG. 5C is a side view of the clamp carriage of FIG. 5A;

FIG. 5D is a front view of the clamp carriage of FIG. 5A;

FIG. 6A illustrates a perspective view of the auxiliary part clamp carriage assembly;

FIG. 6B is a cross sectional view of the clamp carriage of FIG. 6A;

FIG. 6C is a side view of the clamp carriage of FIG. 6A; and

FIG. 6D is a front view of the clamp carriage of FIG. 6A.

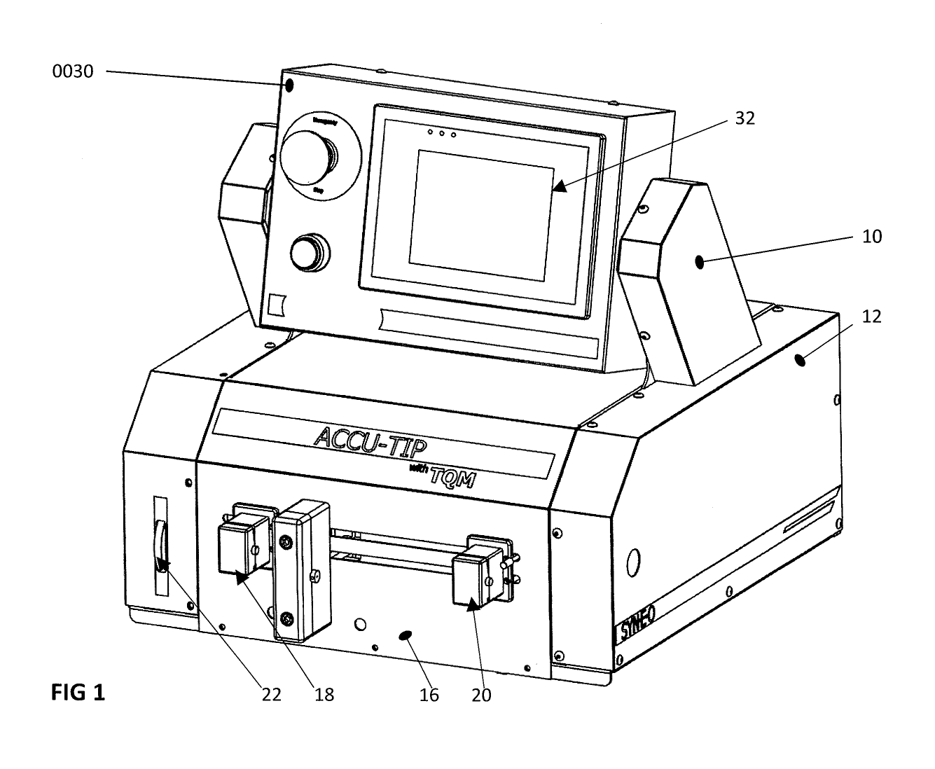

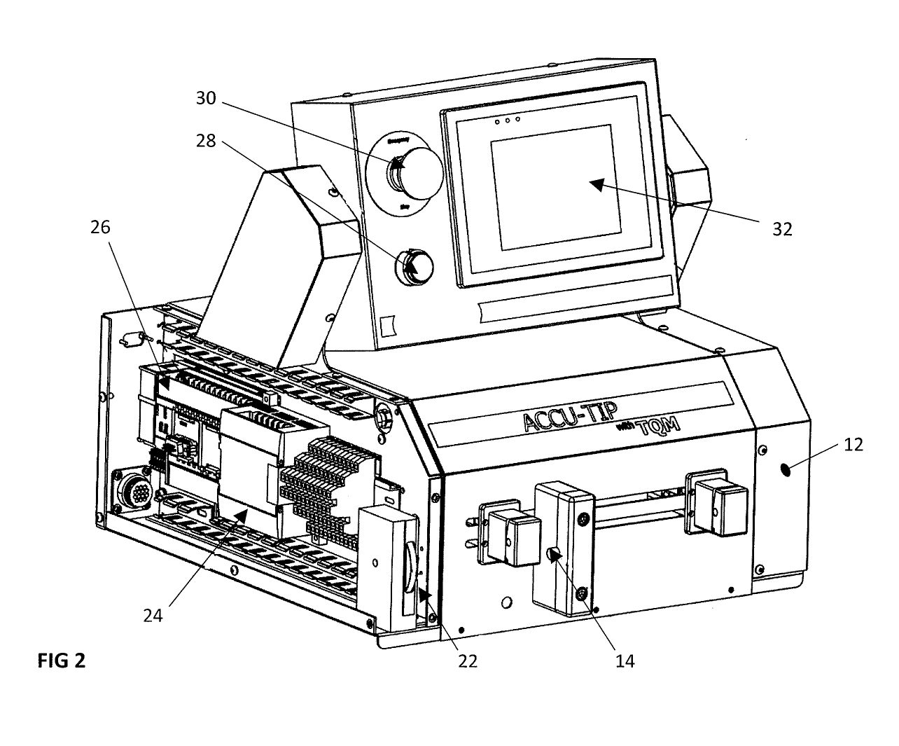

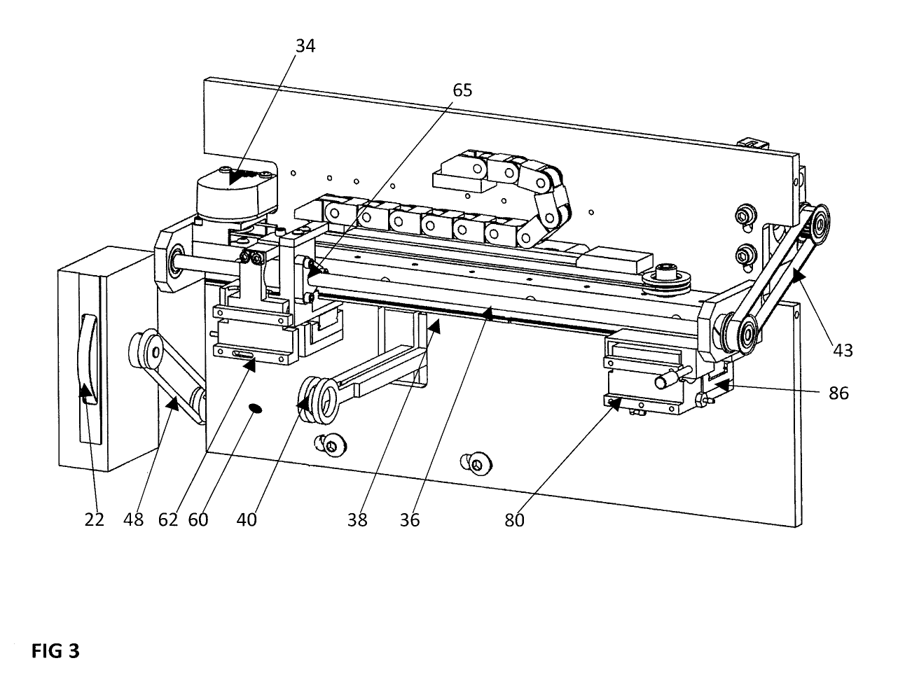

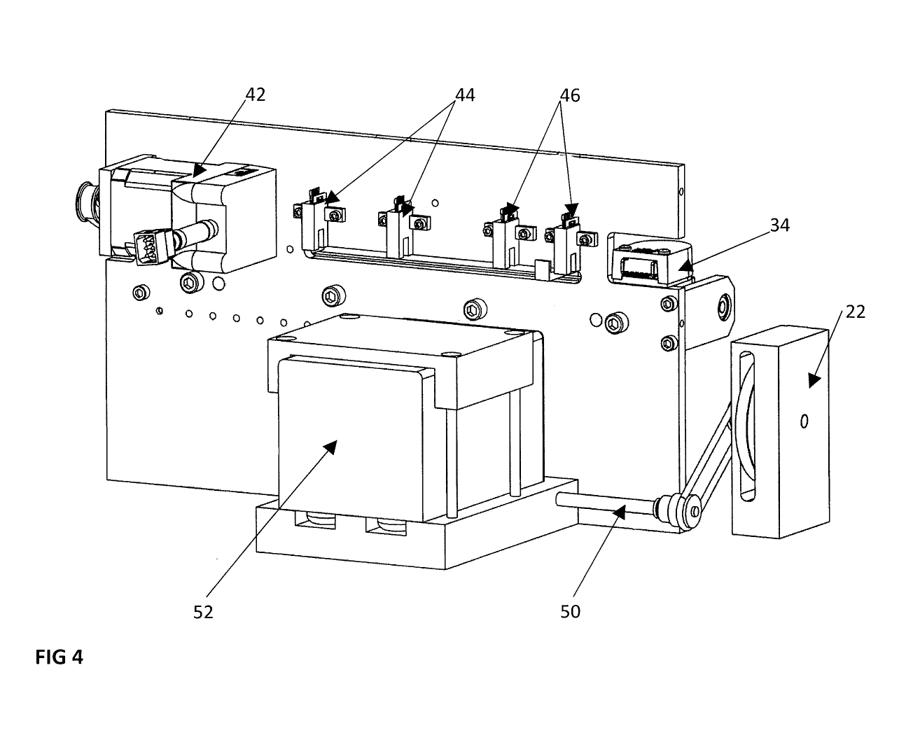

### DETAILED DESCRIPTION OF THE PREFERRED EMBODIMENT

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Disclosed is an apparatus and method to thermally form a smooth profile tip on a polymer tube such as a catheter. The process employs a metal mold that contains the desired form. The tube is inserted partially into the cool mold which is then heated sufficiently to soften the plastic. The tube is then forced further into the mold until the form is completed. The mold and part are cooled to solidify the form and the completed part is removed from the mold. Traditionally the tube is gripped with a clamp and inserted into the mold using an air cylinder and manually controlled air pressure. Automatic process control is limited to temperature and time. In addition to time and temperature control, the instant apparatus and method adds load cell based force feedback and a motorized linear stage with speed control and encoder based position feedback. This additional feedback and control presents flexible options for multiple modes of operation. The control software provides a multi-step process flow where each step includes user programmable force, distance, speed, temperature, and time. Users can define each parameter in each step to produce a desired result.

Referring to Figures in general, set forth are illustrations of the tip forming apparatus 10 of the instant invention. The apparatus has a base 12 with a tip mold and thermocouple 14 positioned along a frontal surface 16. A primary clamp 18 can be used on either side of the mold for left to right, or right to left operation. An auxiliary clamp 20 with pneumatic actuation positioned opposite the primary clamp 18. An RF heater position adjuster 22 is accessible for operating of a temperature controller 24 through a programmable logic controller 26. Operation modes of the apparatus include a run button 28, an emergency stop button 30 and a human-machine interface (HMI) 32.

The clamp axis encoder 34 which operates the clamp axis lead screw 36 and clamp axis linear slide 38 for displacement of the primary clamp. An RF heater 40 provides the part softening temperature.

The primary clamp is positioned by use of a clamp axis drive motor 42 coupled to the axis by a drive belt 43. Limit sensors 44 provide right to left operation control. Limit sensors 46 provide left to right operation control. The RF heater position adjuster 22 provides adjustment through a belt coupler 48 to an RF position adjustment lead screw 50 for control through an RF control head 52.

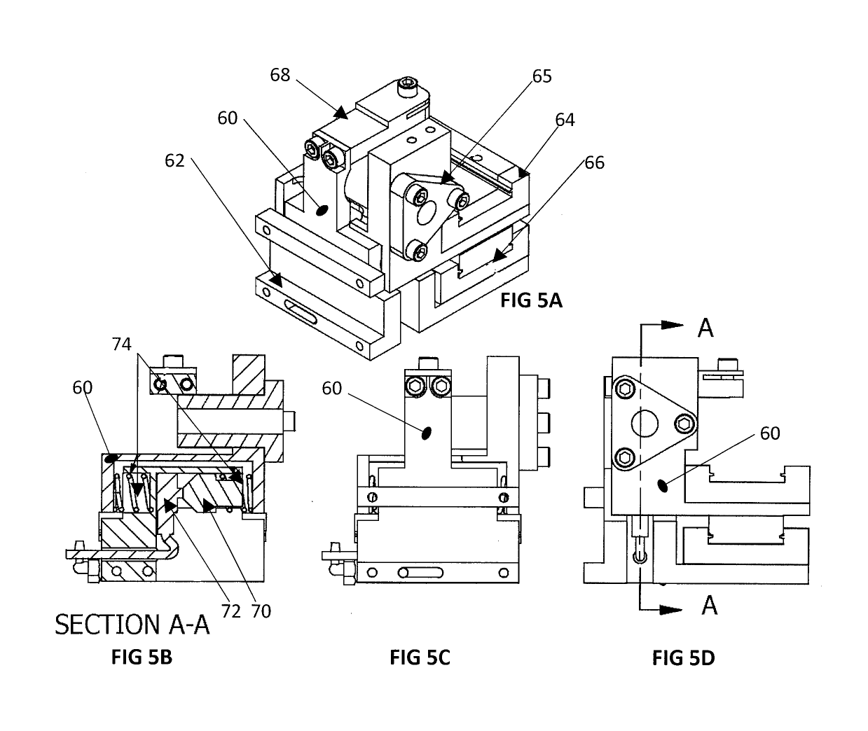

FIGS. 5A-5D discloses the primary part clamp assembly 60. The primary part 60 has a clamp mounting surface 62 and a car 64 linear slide, constructed and arranged to attach to the main rail. The assembly is further defined by a rail 66 linear slide and a preloaded nut 65 lead screw. A bracket encoder cable 68 is positioned along an upper surface. A load cell plunger 70 engages the button load cell 72 with preload springs 74 providing a biasing force.

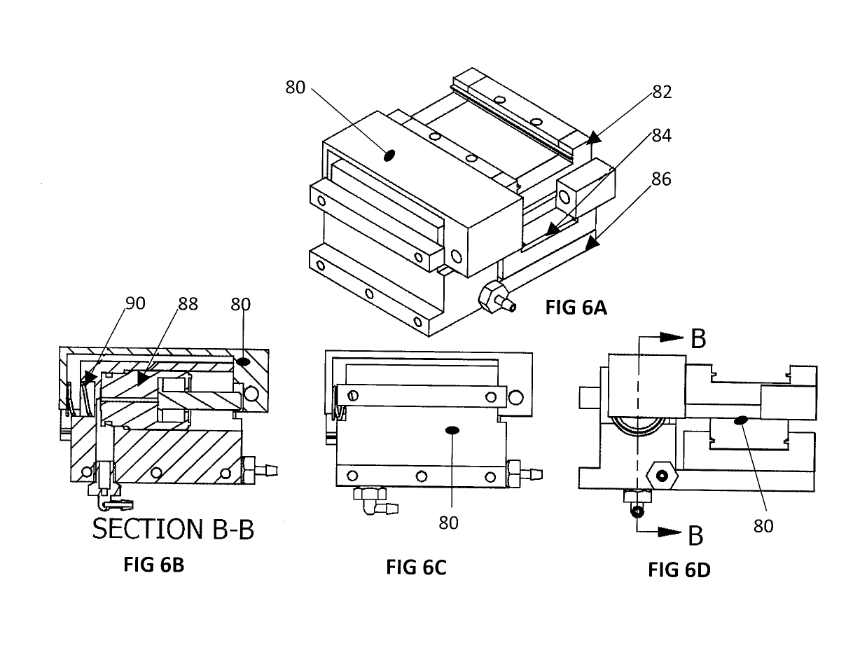

FIGS. 6A-6D discloses the auxiliary part clamp assembly 80. The auxiliary part 80 has a car 82, further defined by the rail linear slide 84 and car linear slide 86. The assembly employs a pneumatic actuator 88 having a return spring 90.

The control software provides a multi-step process flow where each step includes user programmable force, distance, speed, temperature, and time. Users can define each parameter in each step to produce a desired result. The following examples illustrate some of the options available.

Insert with constant force:
1. The operator loads a tube into the gripper and presses start
2. The tube is gripped and inserted into the mold by the motor driven linear stage at the user defined speed until the user defined force is reached as indicated by the load cell. This provides a known and repeatable starting position for each cycle.
3. The RF generator heats the mold by induction until the user defined temperature is reached as indicated by the thermocouple
4. The mold is held at the defined temperature for a user defined time which is controlled by the computer
5. The tube is inserted into the mold at the user defined speed until the user defined force is reached and continues to insert further if the force drops below the set-point. The force is maintained until the user defined time expires
6. The insertion depth provided by the encoder on the linear stage is recorded for comparison to a known good depth. Should a deviation from expected depth be detected a warning message can be displayed.
7. The cooling operation starts and continues until the user defined temperature is reached
8. The linear stage withdraws the tube from the mold while monitoring the force. Should the force exceed acceptable limits a warning message can be displayed.

Insertion to distance:
1. The operator loads a tube into the gripper and presses start
2. The tube is gripped and inserted into the mold by the motor driven linear stage at the user defined speed until the user defined force is reached as indicated by the load cell. This provides a known and repeatable starting position for each cycle.
3. The RF generator heats the mold by induction until the user defined temperature is reached as indicated by the thermocouple
4. The mold is held at the defined temperature for a user defined time which is controlled by the computer
5. The tube is inserted into the mold at the user defined speed until the user defined depth is reached
6. The tube is held in position for a user defined time
7. Steps 5 & 6 can be repeated multiple times with different speed and depth as defined by the user
8. The force provided by the load cell is monitored during tube insertion and compared with user defined maximum force. Should a deviation from expected force be detected a warning message can be displayed.
9. The cooling operation starts and continues until the user defined temperature is reached
10. The linear stage withdraws the tube from the mold while monitoring the force. Should the force exceed acceptable limits a warning message can be displayed.

Users can define many unique cycles by defining force, insertion depth, speed, temperature, and time. Once the process steps are defined the operator need only choose the process from a list (or using a bar code reader) and load the tube and start the process.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An apparatus and method of forming a smooth profile tip on a polymer tube comprising the steps of:
gripping a polymer tube and inserting a tip of said tube into a mold by a motor driven linear stage at a user defined speed;
detecting the amount of force that is applied to the tip of said tube against said mold with a load cell plunger having at least one preload spring until the user defined force is obtained;
heating said mold by induction until a temperature is reached as indicated by a thermocouple;
cooling said mold until a user defined temperature is obtained as indicated by the thermocouple; and
withdrawing the tip from said mold while monitoring force required for removal.

2. The apparatus and method according to claim 1 wherein said polymer tube is a catheter.

3. The apparatus and method according to claim 1 wherein recording said user defined speed and amount of force applied to the tip of said tube against the mold to provide a known and repeatable starting position.

4. The apparatus and method according to claim 1 wherein heating of said mold is with an RF generator.

5. The apparatus and method according to claim 4 wherein the RF generator heats the mold at a defined temperature for a defined time.

6. The apparatus and method according to claim 5 wherein said defined temperature and defined time are user based and controlled by a programmable computer.

7. The apparatus and method according to claim 1 wherein said motor driven linear stage continues to insert the tip further if the load cell detects a force less than a set point.

8. The apparatus and method according to claim 1 wherein including the step of detecting an insertion depth provided by an encoder on said motor driven linear stage and comparing the insertion depth against a measured depth.

9. The apparatus and method according to claim 8 wherein said insertion depth is recorded and a deviation from an expected depth is displayed as a warning message.

10. The apparatus and method according to claim 1 wherein an alarm message is displayed if the force exceeds an acceptable limit when the tip is withdrawn from said mold.

11. The apparatus and method according to claim 1 where said mold is preheated.

12. The apparatus and method according to claim 1 wherein each insertion measured for distance and force is recorded and compared for deviations to detect process anomalies.

13. The apparatus and method according to claim 1 including the step of positioning the tip before a vision inspection module to measure the tip for proper profile dimensions and detect defects.
